(19) United States
(12) Patent Application Publication
(10) Pub. No.: US 2026/0363594 A1
(43) Pub. Date: Nov. 19, 2026

(54) HAND REHABILITATION EVALUATION DEVICE

(71) Applicant: OOPS Limited Company, Tainan (TW)

(72) Inventors: PEI-CHING WU, Tainan (TW); KENG-REN LIN, Tainan (TW); TSAN-HSUN HUANG, Tainan (TW)

(21) Appl. No.: 19/206,719

(22) Filed: May 13, 2025

(51) Int. Cl. A61B 5/22; A61B 90/00

(52) U.S. Cl. CPC A61B 5/225; A61B 90/06; A61B 2090/066; A61B 2562/0261

(57) ABSTRACT

The instant disclosure provides a hand rehabilitation evaluation device. The hand rehabilitation evaluation device includes a handle, a torque dial, and a controller. The handle includes a grip force sensing module and a torque sensing module. The grip force sensing module and the torque sensing module are configured to generate a grip force sensing signal and a torque sensing signal. A second surface of the torque dial includes a plurality of finger force buttons connected to a finger force sensing module, which generates a finger force sensing signal when a finger force button is pressed. The controller receives and calculates various sensing signals to generate grip force data, torque data, and finger force data. The present disclosure integrates a plurality of hand function assessments, thus providing convenient data collection and analysis capabilities.

### CROSS-REFERENCE TO RELATED APPLICATION(S)

The present application claims the benefit of and priority to U.S. Provisional Patent Application Ser. No. 63/646,594, filed on May 13, 2024, entitled “HAND REHABILITATION EVALUATION DEVICE AND DATA TRANSMISSION METHOD”, the contents of which are hereby incorporated herein fully by reference into the present application for all purposes.

### FIELD

The present disclosure generally relates to a technical field of rehabilitative medical devices, more particularly, to a hand rehabilitation evaluation device with multifunctional evaluation capabilities. Such a device is primarily used to assess a hand function of a user, including hand torque, grip force, and individual finger force. It is applicable in various contexts, such as rehabilitative therapy, hand function disorder evaluation, and daily hand strength training.

### BACKGROUND

Hand rehabilitation and functional evaluation play a critical role in the medical field, particularly for patients recovering from hand injuries, post-surgical procedures, or neuromuscular disorders. However, most existing hand rehabilitation evaluation devices available on the market are designed with a single-function focus.

Conventional grip strength measuring devices, such as hand dynamometers, are primarily designed to measure overall hand grip force. These devices typically utilize hydraulic or electronic sensing technologies, where the user applies pressure by squeezing the device to generate a grip force reading. While effective for assessing general grip strength, these devices are limited in that they cannot evaluate individual finger strength or the hand's ability to generate torque, thereby providing insufficient data for patients requiring comprehensive hand function evaluations.

Finger force measuring devices, such as pinch gauges, are specifically developed to assess the strength of individual fingers or specific pinching techniques (e.g., tip pinch, lateral pinch). Although such devices offer more detailed evaluation of finger function, these devices often suffer from cumbersome operation and typically lack data storage capabilities. As a result, medical personnel must manually record each measurement, thus increasing both the time required for evaluation and the potential for error.

Devices for measuring hand torque are relatively rare in the current market. Torque evaluations are often integrated into large-scale rehabilitation systems, such as Isokinetic Dynamometers. These systems are bulky, expensive, and complex to operate, often requiring the assistance of trained professionals, and are therefore unsuitable for routine home-based rehabilitation.

In terms of data management, traditional hand function evaluation devices generally lack a complete data collection and transmission mechanism. Evaluation data usually need to be manually recorded in medical records by medical personnel, making long-term tracking and analysis difficult. Even though some modern devices have data storage capabilities, their data transmission methods are still mostly limited to wired connections, which are not convenient to use.

Furthermore, existing devices often fail to consider ergonomic factors, especially the differences in left and right hand usage. Most devices adopt a unified design without accounting for the anatomical differences between the left and right hands, which may lead to discomfort or measurement inaccuracies during evaluation for certain users.

Finally, traditional evaluation devices lack intuitive visual feedback mechanisms. Users are unable to immediately understand their exertion levels or test results, which reduce their sense of participation and motivation during the rehabilitation process. For patients requiring long-term hand rehabilitation, the lack of an immediate feedback mechanism may lead to reduced rehabilitation compliance and affect the overall rehabilitation effect.

In summary, current technologies for hand rehabilitation evaluation suffer from limitations, such as scattered functions, inefficient data management, and non-user-friendly design. There is a pressing need for an innovative device that integrates multi-functional evaluation capabilities with convenient data handling and ergonomic features to improve the effectiveness and user experience of hand rehabilitation evaluations.

### SUMMARY

In view of the foregoing, there is a need to provide a device capable of collecting multiple types of hand function evaluation data within a single apparatus, thereby offering a comprehensive basis for users or rehabilitation professionals to formulate subsequent rehabilitation strategies and treatment plans.

According to a first aspect of the present disclosure, a hand rehabilitation evaluation device configured to be held by a hand of a user for collecting hand function evaluation data is provided. The hand rehabilitation evaluation device includes a handle, a torque dial, and a controller. The handle includes a grip force sensing module and a torque sensing module, one side of the handle comprising a first grip actuator and another side of the handle comprising a second grip actuator, each of the first grip actuator and the second grip actuator being connected to the grip force sensing module, the grip force sensing module being configured to generate a grip force sensing signal in response to a grip pressure applied to the first grip actuator and second grip actuator. The torque dial includes a first surface rotatably connected to a first end portion of the handle, the torque dial being connected to the torque sensing module, the torque dial being connected to the torque sensing module, the torque sensing module being configured to generate a torque sensing signal in response to a rotation of the torque dial, the torque dial further comprising a second surface disposed opposite the first surface, the first surface located at a bottom of the torque dial, the second surface being provided with a plurality of finger force buttons, each of the plurality of finger force buttons being coupled to a finger force sensing module, the finger force sensing module being configured to generate a plurality of finger force sensing signals in response to a plurality of pressures applied to the plurality of finger force buttons. The controller including a processor, the controller being disposed within the handle, the processor is electrically connected to the torque sensing module, the grip force sensing module, and the finger force sensing module, the processor being configured to: receive the grip force sensing signal, the torque sensing signal, and the plurality of finger force sensing signals; and generate a grip force data, a torque data, and a finger force data based on the grip force sensing signal, the torque sensing signal, and the plurality of finger force sensing signals.

In an implementation of the first aspect of the present disclosure, the controller includes a storage module coupled to the processor, the storage module being configured to store the grip force data, the torque data, and the finger force data generated by the processor.

In another implementation of the first aspect of the present disclosure, the torque sensing module includes a spring shaft member and a rotary-type variable resistor, the rotary-type variable resistor being disposed within the spring shaft member, when the torque dial rotates relative to the handle, the torque dial is configured to cause the spring shaft member to synchronously rotate with the torque dial, thereby causing a resistance value of the rotary-type variable resistor to change correspondingly with a change of a rotation angle, and the torque sensing module is configured to convert a change of the resistance value into the torque sensing signal, the processor is further configured to calculate a torsion amplitude and a torque based on the torque sensing signal, thereby generating the torque data.

In another implementation of the first aspect of the present disclosure, the finger force sensing module includes a micro strain gauge-based sensor, each of the plurality of finger force buttons is configured to drive the micro strain gauge-based sensor in response to a pressure applied to a corresponding finger force button of the plurality of finger force buttons, the micro strain gauge-base sensor is configured to sense a pressure conducted by each of the plurality of finger force buttons and to generate the plurality of finger force sensing signals, the finger force sensing module is configured to transmit the plurality of finger force sensing signals to the controller for calculation by the processor to generate the finger force data.

In another implementation of the first aspect of the present disclosure, each of the plurality of finger force buttons is internally provided with a light-emitting element, each of the plurality of finger force buttons is configured to emit a light source in response to the pressure applied to the corresponding finger force button, and a brightness or a color of each of a plurality of light sources emitted, including the light source, by the light-emitting element is determined based on the pressure received by each of the plurality of finger force buttons.

In another implementation of the first aspect of the present disclosure, the grip force sensing module includes a strain gauge-based sensor, the strain gauge-based sensor is configured to sense the grip pressure in response to the grip pressure applied to the first grip actuator and the second grip actuator to generate the grip force sensing signal, and the grip force sensing module is further configured to transmit the grip force sensing signal to the controller for calculation by the processor to generate the grip force data.

In another implementation of the first aspect of the present disclosure, a second end portion of the handle is provided with a charging transmission port connected to the controller, and the charging transmission port is configured to charge the hand rehabilitation evaluation device and to transmit data.

In another implementation of the first aspect of the present disclosure, the hand rehabilitation evaluation device is detachably connected to a connector, the connector includes a connection plug, and when the hand rehabilitation evaluation device is connected to the connector, the connection plug engages with the charging transmission port for charging the hand rehabilitation evaluation device and transmitting the data

In another implementation of the first aspect of the present disclosure, an outer portion of the handle is provided with a display screen and a torque indicator light bar, both of the display screen and the torque indicator light bar are electrically connected to the controller, the display screen is configured to display one of the grip force data, the torque data, and the finger force data, and the torque indicator light bar is configured to illuminate based on a rotation direction of the torque dial.

In another implementation of the first aspect of the present disclosure, the hand rehabilitation evaluation device further includes a wireless transmission module connected to the controller, and the wireless transmission module is configured to externally transmit the grip force data, the torque data, and the finger force data stored in the storage module.

In another implementation of the first aspect of the present disclosure, the connector is provided with a wireless communication module connected to the controller, and the wireless connection module is configured to externally transmit the grip force data, the torque data, and the finger force data stored in the storage module.

In another implementation of the first aspect of the present disclosure, a number of the plurality of finger force buttons is six.

### BRIEF DESCRIPTION OF THE DRAWINGS

The present description will be better understood from the following detailed description when read in light of the accompanying drawings, where:

FIG. 1 illustrates a perspective view of a hand rehabilitation evaluation device according to an example implementation of the present disclosure.

FIG. 2 is a front view of the hand rehabilitation evaluation device according to an example implementation of the present disclosure.

FIG. 3 is a top view of a torque dial according to an example implementation of the present disclosure.

FIG. 4 is a schematic view illustrating a torque rotation of the torque dial according to an example implementation of the present disclosure.

FIG. 5 is a block diagram illustrating a controller and various sensing modules of the hand rehabilitation evaluation device according to an example implementation of the present disclosure.

FIG. 6 is a schematic view of a second end potion of the hand rehabilitation evaluation device according to an example implementation of the present disclosure.

FIG. 7 is a schematic view illustrating an operation of the torque dial according to an example implementation of the present disclosure.

FIG. 8 is a schematic exploded view illustrating disassembling and assembling of a handle and the torque dial according to an example implementation of the present disclosure.

FIG. 9 is another schematic exploded view illustrating the disassembling and assembling of the handle and the torque dial according to an example implementation of the present disclosure.

FIG. 10 is a schematic view illustrating an operation of the hand rehabilitation evaluation device according to an example implementation of the present disclosure.

FIG. 11 is a schematic view illustrating an assembly of the hand rehabilitation evaluation device and a connector according to an example implementation of the present disclosure.

FIG. 12 is a schematic diagram illustrating a use of the hand rehabilitation evaluation device with the connector according to an example implementation of the present disclosure.

FIG. 13 is a schematic view of the hand rehabilitation evaluation device according to an example implementation of the present disclosure.

FIG. 14 is a top schematic view of the torque dial according to an example implementation of the present disclosure.

### DETAILED DESCRIPTION

The detailed description provided below in connection with the appended drawings is intended as a description of the present examples and is not intended to represent the only forms in which the present examples may be constructed or utilized. The description sets forth the functions of the examples and the sequence of steps for constructing and operating the examples. However, the same or equivalent functions and sequences may be accomplished by different examples.

For convenience, certain terms employed in the specification, examples, and appended claims are collected here. Unless otherwise defined herein, scientific, and technical terminologies employed in the present disclosure shall have the meanings that are commonly understood and used by one of ordinary skill in the art. Also, unless otherwise required by context, it will be understood that singular terms shall include plural forms of the same, and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Terms such as “at least one embodiment”, “one embodiment”, “multiple embodiments”, “different embodiments”, “some embodiments”, “present embodiment”, and the like may indicate that an embodiment of the present disclosure so described may include a particular feature, structure, or characteristic, but not every possible embodiment of the present disclosure must include a particular feature, structure, or characteristic. Furthermore, repeated use of the phrases “in one embodiment”, “in the embodiment”, and so on does not necessarily refer to the same embodiment, although they may be identical. Furthermore, the use of phrases such as “embodiments” in connection with “the present disclosure” does not imply that all embodiments of the present disclosure necessarily include a particular feature, structure, or characteristic, and should be understood as “at least some embodiments of the present disclosure” include the particular feature, structure, or characteristic described.

Additionally, for the purposes of explanation and non-limitation, specific details such as functional entities, techniques, protocols, standards, and the like are set forth for providing an understanding of the described technology. In other examples, detailed disclosure of well-known methods, technologies, systems, architectures, and the like are omitted so as not to obscure the disclosure with unnecessary details.

The terms “first”, “second”, and “third” in the description of the present disclosure and the above-mentioned drawings are used to distinguish different objects, rather than to describe a specific order.

Furthermore, the term “comprising” and any variations thereof are intended to cover non-exclusive inclusions and may refer to “including but not necessarily limited to”, which specifically indicates open-ended inclusion or membership in the so-described combination, group, series, and the equivalent. For example, a process, method, system, product, or device that includes a series of steps or modules is not limited to the listed steps or modules, but optionally also includes steps or modules that are not listed, or optionally also includes other steps or modules that are inherent to those processes, methods, products, or devices.

The implementations of the present disclosure are described below with reference to the accompanying drawings.

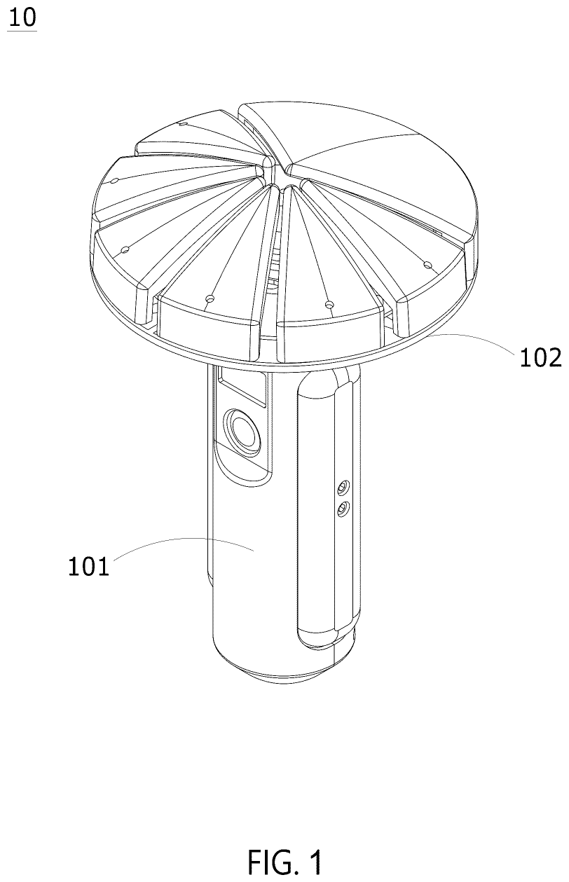

FIG. 1 illustrates a perspective view of a hand rehabilitation evaluation device according to an example implementation of the present disclosure.

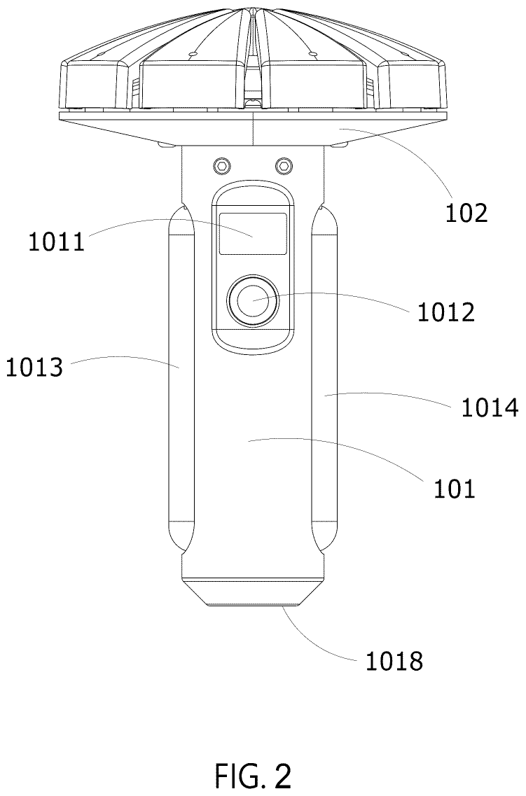

Referring to FIG. 1, the hand rehabilitation evaluation device 10 primarily includes a handle 101 and a torque dial 102. The torque dial 102 is rotatably connected to a first end portion 1019 of the handle 101 (referring to FIG. 2). The handle 101 is configured in an elongated columnar shape to provide a comfortable gripping experience for the user, and may alternatively be implemented in a non-cylindrical form. The torque dial 102 has a generally disk-shaped structure and is rotatably coupled to the handle 101 through a specially designed rotational connection mechanism, allowing the torque dial rotates in a clockwise direction or a counterclockwise direction. The rotational angle may correspond to various levels of torque demand. The torque dial 102 may also adopt a non-disk-shaped design, such as a mushroom shape, spherical shape, or other irregular forms, and the appearance of the torque dial 102 102 is not limited herein. When the user rotates the torque dial 102 to the left or right, the above action simulates common twisting motions encountered in daily activities, such as opening bottle caps or turning doorknobs, thereby facilitating the evaluation and training of the user's rotational hand function. The integrated design of the handle 101 and the torque dial 102 enables the device to integrate a plurality of hand function evaluations into a single device, thereby substantially enhancing usability and evaluation efficiency.

FIG. 2 is a front view of the hand rehabilitation evaluation device according to an example implementation of the present disclosure.

Referring to FIG. 2. The handle 101 is configured in an elongated columnar shape to facilitate user grip. The first end portion 1019 of the handle 101 is connected to the torque dial 102, and a second end portion 1018 is at the bottom of the handle 101. A display screen 1011 and an operation button 1012 are disposed on the front side of the handle 101. The operation button 1012 is configured to select functions and perform control operations, while the display screen 1011 is configured to present the user interface and display real-time measurement data. A first grip force actuator 1013 is disposed on one side of the handle 101, and a second grip force actuator 1014 is disposed on another side of the handle 101, where the one side of the handle 101 and another side of the handle are opposite lateral sides. Furthermore, the first grip force actuator 1013 and the second grip force actuator 1014 are used for measuring the user's grip strength. The user may grasp the handle 101 with one hand and simultaneously apply pressure on the first grip force actuator 1013 and the second grip force actuator 1014 using the thumb and other fingers to perform grip force measurements.

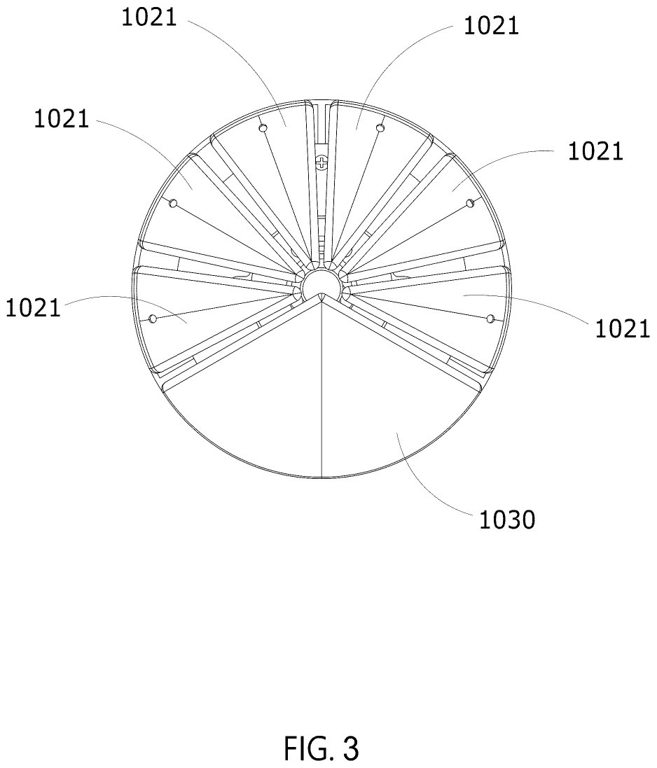

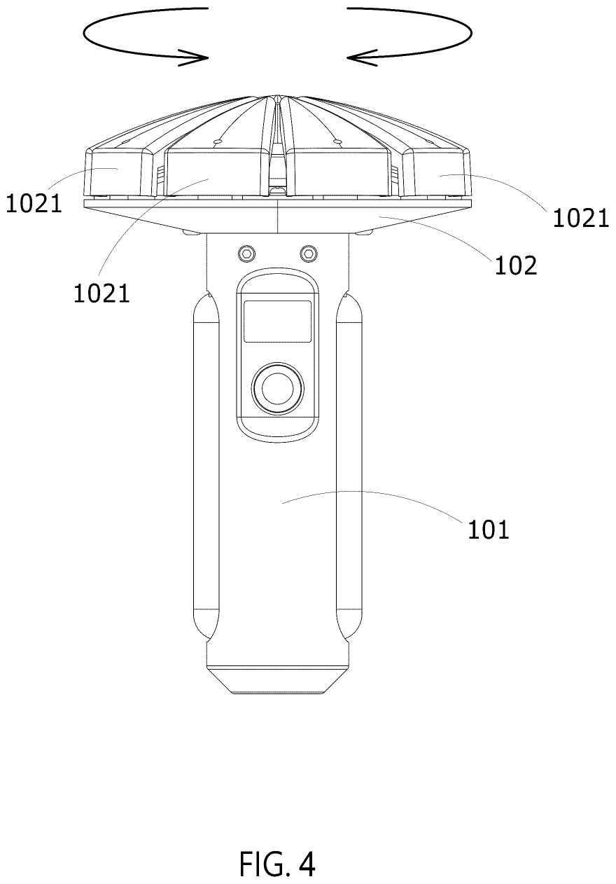

FIG. 3 is a top view of a torque dial according to an example implementation of the present disclosure. FIG. 4 is a schematic view illustrating a torque rotation of the torque dial according to an example implementation of the present disclosure.

Referring to FIG. 3, the torque dial 102 is disc-shaped and is provided with a plurality of finger force buttons 1021 disposed on a second surface 1030 of the torque dial 102, and the plurality of finger force buttons 1021 is configured to sense a plurality of pressures applied by fingers on the plurality of finger force buttons 1021 for measuring a plurality of finger forces from an user. In addition, the user may grasp the torque dial 102 and perform rotational movements in either the clockwise or counterclockwise direction. The rotational movement of the torque dial 102 is illustrated in FIG. 4. Referring to FIG. 4, arrows in FIG. 4 indicate that the torque dial 102 could rotate in both clockwise and counterclockwise directions. During use, the user may hold the handle 101 with one hand and place the other hand on the torque dial 102 such that the palm is slightly arched, and the fingers apply the plurality of pressures to the plurality of finger force buttons 1021. In this configuration, by rotating the palm leftward or rightward, the torque dial 102 could be rotated relative to the handle 101.

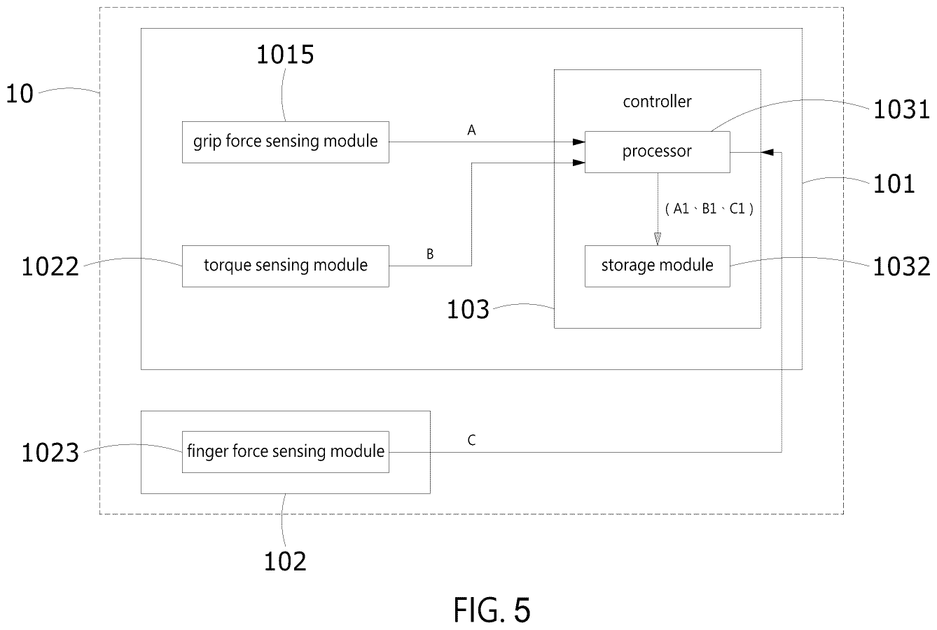

FIG. 5 is a block diagram illustrating a controller and various sensing modules of the hand rehabilitation evaluation device according to an example implementation of the present disclosure.

Please referring to FIG. 5 and also referring to FIGS. 1 to 4. The handle 101 is internally provided with a grip force sensing module 1015 and a torque sensing module 1022. Each of the first grip force actuator 1013 and the second grip force actuator 1014 is connected to the grip force sensing module 1015. When both of the first grip force actuator 1013 and second grip force actuator 1014 are simultaneously pressed, the grip force sensing module 1015 generates a grip force sensing signal A. The torque dial 102 is connected to the torque sensing module 1022. When the torque dial 102 is rotated in the clockwise or counterclockwise direction, the torque sensing module 1022 detects the rotational behavior of the torque dial 102 and generates a torque sensing signal B. Furthermore, each of the plurality of finger force buttons 1021 is connected to a finger force sensing module 1023. When the plurality of finger force buttons 1021 are pressed, the finger force sensing module 1023 generates a finger force sensing signal C. In addition, the hand rehabilitation evaluation device 10 further includes a controller 103 including a processor 1031 and a storage module 1032, where the controller 103 is disposed within the handle 101. The processor 1031 is electrically connected to the torque sensing module 1022, the grip force sensing module 1015, and the finger force sensing module 1023, for receiving the grip force sensing signal A from the grip force sensing module 1015, the torque sensing signal B from the torque sensing module 1022, and the finger force sensing signal C from the finger force sensing module 1023. The processor 1031 performs calculation and processing on each of the received signals (e.g., the grip force sensing signal A, the torque sensing signal B and the finger force sensing signal C) to generate grip force data A1, torque data B1, and finger force data C1. The storage module 1032 is electrically connected to the processor 1031 and serves to store the various data (e.g., the grip force data A1, the torque data B1, and the finger force data C1) generated by the processor 1031. During measurement operations, the controller 103 receives and processes the sensing signals from each of the sensing modules, automatically calculates one of the grip force data A1, the torque data B1, or the finger force data C1, and displays the results on the display screen 1011. Simultaneously, the grip force data A1, the torque data B1, and the finger force data C1 are stored in the storage module 1032 for subsequent analysis. In some implementations, the finger force sensing module 1023 includes micro strain gauge-based sensor. When the finger force buttons 1021 are pressed, the micro strain gauge-based sensor detects the pressure level and generate the finger force sensing signal C, which is transmitted to the controller 103 and processed by the processor 1031 to generate the finger force data C1. In some implementations, the grip force sensing module 1015 includes strain gauge-based sensor. When the user simultaneously presses the first grip force actuator 1013 and the second grip force actuator 1014, the strain gauge-based sensor detects the grip force and generate the grip force sensing signal A, which is transmitted to the controller 103 and processed by the processor 1031 to generate the grip force data A1.

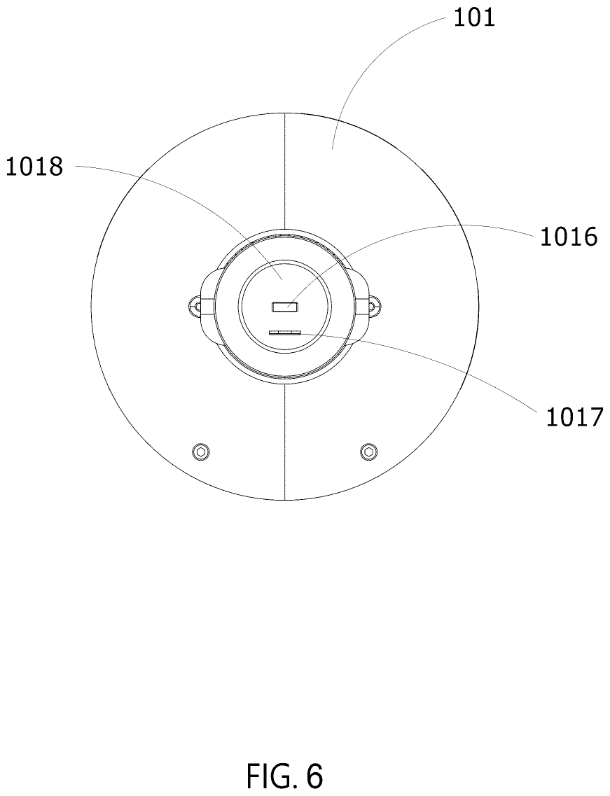

FIG. 6 is a schematic view of a second end potion of the hand rehabilitation evaluation device according to an example implementation of the present disclosure.

Referring to FIG. 6 and FIG. 5. As shown in FIG. 6, the second end portion 1018 of the handle 101 is provided with a charging and data transmission port 1016 and a memory card slot 1017. Each of the charging and data transmission port 1016 and the memory card slot 1017 is connected to the controller 103. The charging and data transmission port 1016 is configured to provide charging and data transmission for the hand rehabilitation evaluation device 10. The memory card slot 1017 is configured to receive a memory card with storage functionality, serving as a medium for data storage.

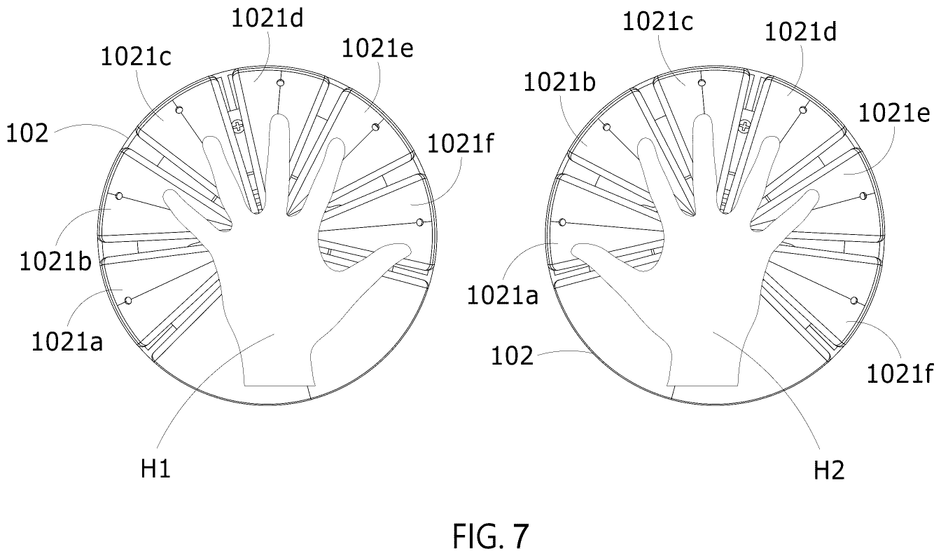

FIG. 7 is a schematic view illustrating an operation of the torque dial according to an example implementation of the present disclosure.

As shown in FIG. 7, the torque dial 102 includes a plurality of finger force buttons 1021. Preferably, the number of the plurality of finger force buttons 1021 is six (1021a, 1021b, 1021c, 1021d, 1021e, and 1021f shown in the FIG. 7). Due to the structural differences between the left and right hands, particularly in the position of the thumb, the six finger force buttons 1021 are arranged ergonomically on the torque dial 102 to enable comfortable operation by both left and right hands. As the left hand H1 and the right hand H2 shown in FIG. 7, the position of the thumb on the palm is obviously different from the other four fingers. For example, if the thumb of the right hand H2 is placed on finger force button 1021b, the index finger of the right hand H2 is placed on finger force button 1021c, the middle finger of the right hand H2 is placed on finger force button 1021d, the ring finger of the right hand H2 is placed on finger force button 1021e, and the little finger of the right hand H2 is placed on finger force button 1021f, the hand and wrist would need to twist uncomfortably. Therefore, having six finger force buttons allows for ergonomic positioning of all five fingers of either left hand H1 or right hand H2 to perform individual finger force measurements. In some implementations, the number of finger force buttons 1021 could be reduced to four (e.g., only 1021a, 1021b, 1021c, and 1021d are provided). In such a case, the thumb of the left hand H1 may be placed on 1021d, the index finger of the left hand H1 may be placed on 1021c, and middle finger of the left hand H1 may be placed on 1021b, while the thumb of the right hand H2 may be placed on 1021a, the index finger of the right hand H2 may be placed on 1021b, and middle finger of the right hand H2 may be placed on and 1021c to perform finger force testing. It should be noted that the size of the torque dial 102 may be adjusted based on user requirements. The relative size between the hand and the torque dial 102 as shown in FIG. 7 is not intended to limit the scope of the present disclosure. For example, the torque dial 102 may be designed with a size suitable for operation when the user's hand is in a semi-open position, thereby enabling optimal finger force evaluation.

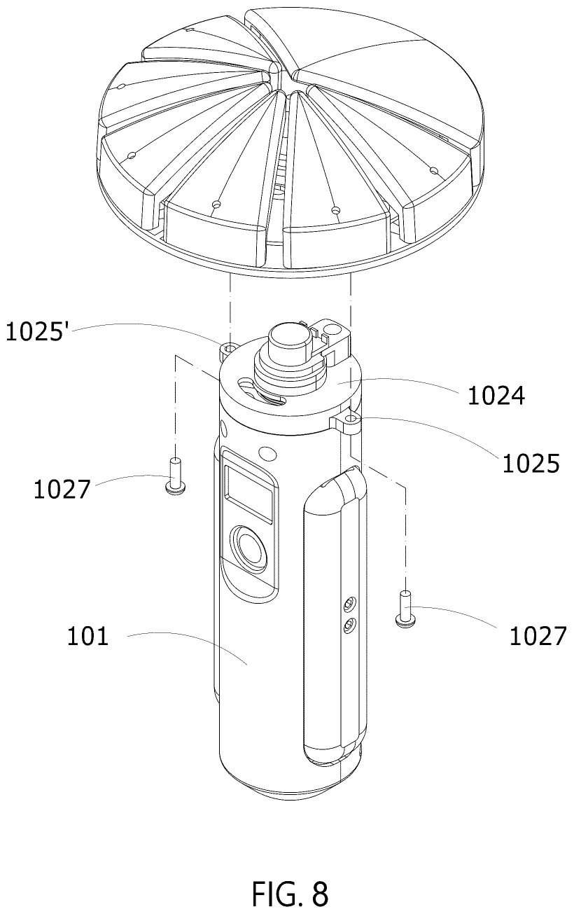

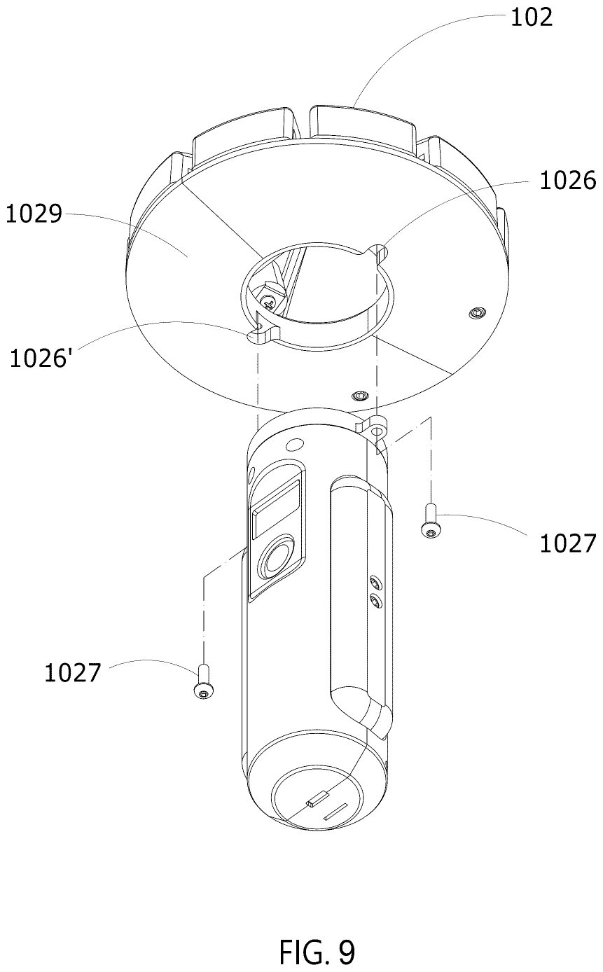

FIG. 8 is a schematic exploded view illustrating disassembling and assembling of a handle and the torque dial according to an example implementation of the present disclosure. FIG. 9 is another schematic exploded view illustrating the disassembling and assembling of the handle and the torque dial according to an example implementation of the present disclosure.

As shown in FIG. 8, the torque sensing module 1022 disposed within the handle 101 (not illustrated in FIG. 8) primarily includes a spring shaft member 1024 and a rotary-type variable resistor. The rotary-type variable resistor is mounted within the spring shaft member 1024, and the spring shaft member 1024 includes two sides which are opposing to each other. Each of the two sides of the spring shaft member 1024 is formed with a pair of fixing holes (1025, 1025′). Pleas also referring to FIG. 8 and FIG. 9, the first surface 1029 of the torque dial 102 is formed with corresponding fixing holes (1026, 1026′) that align with the fixing holes (1025, 1025′) of the spring shaft member 1024. Each of the fixing holes (1025, 1025′) could receive fixing screws 1027 which pass through and secure into the corresponding fixing holes (1026, 1026′), thereby integrally fixing the torque dial 102 to the handle 101.

Referring to FIG. 8 and FIG. 9 in conjunction with FIG. 5, when the torque dial 102 rotates relative to the handle 101, the spring shaft member 1024 rotates synchronously with the torque dial 102, thereby causing a resistance value of the rotary-type variable resistor to change correspondingly with a change of a rotation angle. The torque sensing module 1022 is configured to convert a change of the resistance value into the torque sensing signal B, which is then transmitted to the controller 103. The processor 1031 of the controller 103 processes the signal to calculate a torsion amplitude and a torque applied to the torque dial 102, thereby generating the torque data B1.

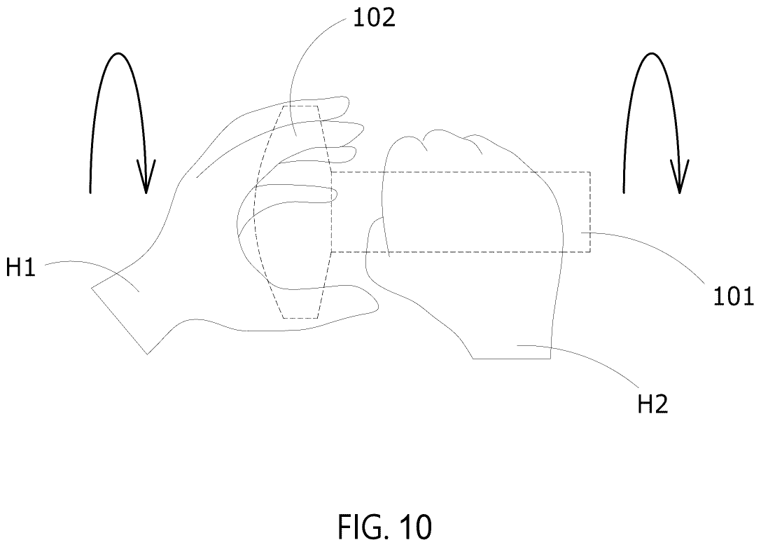

FIG. 10 is a schematic view illustrating an operation of the hand rehabilitation evaluation device according to an example implementation of the present disclosure.

Referring to FIG. 10 and FIGS. 1 to 4, the operation of the hand rehabilitation evaluation device 10 is designed to simulate common daily activities such as opening a jar, while simultaneously enabling the evaluation of a plurality of hand functions. As shown in FIG. 10, a user may grasp the handle 101 with the palm of the right hand H2, while simultaneously grasping the torque dial 102 with the palm of the left hand H1. The fingers of the hand grasping the torque dial 102 are positioned on the corresponding finger force buttons 1021. Meanwhile, the right hand H2, which is grasping the handle 101, is in a fully closed grasping position, whereas the left hand H1, which is grasping the torque dial 102, is in a semi-open position. During operation, the user may exert force on both the first grip force actuator 1013 and the second grip force actuator 1014 using the palm of the hand holding the handle 101, while simultaneously pressing the finger force buttons 1021 with the fingers of the palm of the hand grasping the torque dial 102. The user may then twist both hands in opposite directions (i.e., in a clockwise or counterclockwise direction). This operational configuration allows the evaluation process to more closely resemble natural movements encountered in daily life, thereby enhancing the practical applicability and relevance of the evaluation.

As shown in FIG. 10, the structural design of the handle 101 and the torque dial 102 takes ergonomic principles into account. The handle 101 is configured as a cylindrical structure, which is suitable for evaluating gripping force. The torque dial 102 is shaped to allow the user's palm to remain in a semi-open position, which is particularly advantageous for assessing finger force. A bidirectional rotational structure is disposed between the torque dial 102 and the handle 101, allowing the hand rehabilitation evaluation device to record torque values and rotational angles in both clockwise and counterclockwise directions. With this design, the user is able to perform a single jar-opening-like motion while simultaneously evaluating three distinct hand function parameters. Notably, the torque measurement mechanism of the present disclosure adopts a physically rotatable structure (e.g., based on a cam-lock mechanism), which differs from conventional torque gauges that typically do not permit actual rotation. The torque measurement mechanism of the present disclosure provides a more realistic evaluation experience that better reflects actual usage scenarios. Furthermore, the hand rehabilitation evaluation device is capable of simultaneously recording various combinations of data, including: a full combination of “grip force on one hand+finger force on the other hand+bilateral torque,” or partial combinations such as “grip force on one hand+bilateral torque”, “grip force on one hand +finger force on the other hand,” or “finger force on one hand+bilateral torque,” thereby accommodating different evaluation needs.

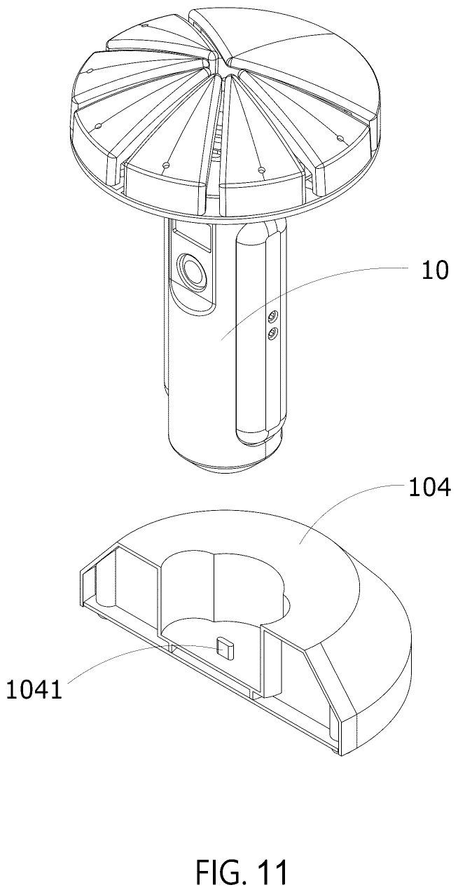

FIG. 11 is a schematic view illustrating an assembly of the hand rehabilitation evaluation device and a connector according to an example implementation of the present disclosure.

Please refer to FIG. 11 and FIG. 6. The hand rehabilitation evaluation device 10 of the present disclosure further includes a connector 104. The connector 104 includes a connection plug 1041. The hand rehabilitation evaluation device 10 is detachably connectable to the connector 104 such that the connection plug 1041 mates with the charging transmission port 1016. Through this connection, charging and data transmission could be performed through the charging transmission port 1016. When the hand rehabilitation evaluation device 10 is placed onto the connector 104, the hand rehabilitation evaluation device is not only capable of being charged but could also simultaneously transmit data. Accordingly, the hand rehabilitation evaluation device 10 is configured to transmit data stored in the storage module 1032 during the charging process.

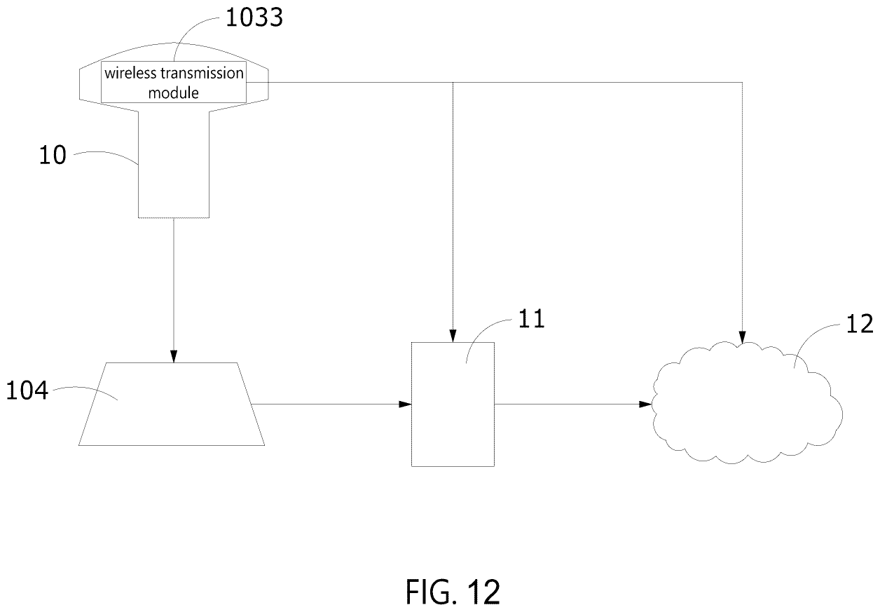

FIG. 12 is a schematic diagram illustrating a use of the hand rehabilitation evaluation device with the connector according to an example implementation of the present disclosure.

Please refer to FIG. 12 and FIG. 5. After the hand rehabilitation evaluation device 10 is coupled with the connector 104, the data stored in the storage module 1032 could be transmitted through the connector 104 to an external electronic device 11, such as a smartphone, which has a dedicated application (APP) pre-installed. The data transmission between the connector 104 and the electronic device 11 may be performed through a wired or wireless connection, which is not limited herein. Once the electronic device 11 receives the data, the electronic device 11 may further upload the data to a cloud server 12 for analysis and storage. In some implementations, the electronic device 11 running the dedicated APP may periodically convert the user's evaluation data into visualized charts, indicating key metrics such as maximum grip force and duration, maximum torque and rotation angle, maximum finger force and tapping frequency. This visualized information could be reviewed by both the user and a therapist, allowing the therapist to evaluate the user's condition and arrange appropriate training plans accordingly. The APP may also provide an interactive interface, such as a virtual pet in the form of an electronic mushroom, that encourages the user to perform massage-like operations on the hand rehabilitation evaluation device 10 in order to feed, treat, or train the virtual pet. In some implementations, the interactive interface may exist solely between the hand rehabilitation evaluation device 10 and the user, functioning as a standalone virtual pet system. In this configuration, the user is guided through interaction using a display screen or indicator lights provided on the hand rehabilitation evaluation device 10 itself or on the connector, the hand rehabilitation evaluation device 10 is without requiring a direct connection to the application (APP). Furthermore, the hand rehabilitation evaluation device 10 may also function as a game controller joystick, the hand rehabilitation evaluation device 10 is connectable to a gaming console. The hand rehabilitation evaluation device 10 could be used to perform game operations, such as rotating the torque dial 102, pressing the plurality of finger force buttons 1021, or grasping the handle 101, which could all be used as game operation methods.

In some implementations, the hand rehabilitation evaluation device 10 further includes a wireless transmission module 1033. The controller 103 is connected to the wireless transmission module 1033, such that the various data stored in the storage module 1032 may be transmitted externally through the wireless transmission module 1033. For example, the data may be wirelessly transmitted directly to the electronic device 11 or the cloud server 12. In this mode, the hand rehabilitation evaluation device 10 is optimally equipped with a wireless communication module such that, upon powering on, the hand rehabilitation evaluation device could automatically establish a connection with the application (APP).

In some implementations, the data generated during grip force, finger force, and torque operations is recorded as shown in Table 1. Each time the hand rehabilitation evaluation device 10 is activated, a data file is generated to record the activity of that session in a format such as shown in the table below. The sampling frequency is at least once every three seconds.

TABLE 1

| | grip | finger | finger | finger | finger | finger | finger | |
|---|---|---|---|---|---|---|---|---|
| Time | force | force 1 | force 2 | force 3 | force 4 | force 5 | force 6 | torque |
| 2023/12/28 | 10 kg | 0 | 0 | 4 kg | 0 | 0 | 0 | 33 kg |
| 19:00:00 | | | | | | | | |
| 2023/12/28 | 11 kg | 3 kg | 0 | 5 kg | 0 | 0 | 0 | 35 kg |
| 19:00:01 | | | | | | | | |
| 2023/12/28 | 12 kg | 0 | 0 | 2 kg | 0 | 0 | 0 | 35 kg |
| 19:00:02 | | | | | | | | |
| 2023/12/28 | 9 kg | 0 | 0 | 1 kg | 0 | 0 | 0 | 35 kg |
| 19:00:03 | | | | | | | | |
| 2023/12/28 | 15 kg | 0 | 0 | 1 kg | 0 | 0 | 0 | 0 |
| 19:00:04 | | | | | | | | |

In Table 1, the grip force is the grip force data A1 generated by the controller 103 after detecting the grip force sensing signal A through the grip force sensing module 1015. The finger forces 1 to 6 correspond to the pressing positions of each of the plurality of finger force buttons (1021a, 1021b, 1021c, 1021d, 1021e, 1021f), and the finger force data C1 generated by the controller 103 after detecting the finger force sensing signal C through the finger force sensing module 1023. The torque is the torque data B1 generated by the controller 103 after detecting the torque sensing signal B through the torque sensing module 1022.

In some implementations, after the electronic device 11 with the dedicated APP installed receives the file, the system records the file content in a data page, with each case having an independent data page. For example, as shown in Table 2.

TABLE 2

| | | | finger | finger | finger | finger | finger | finger | |
|---|---|---|---|---|---|---|---|---|---|
| | | grip | force | force | force | force | force | force | |
| Date | Time | force | 1 | 2 | 3 | 4 | 5 | 6 | torque |
| 2023 Dec. 28 | 19:00:00 | 10 kg | 0 | 0 | 4 kg | 0 | 0 | 0 | 33 kg |
| 2023 Dec. 28 | 19:00:01 | 11 kg | 3 kg | 0 | 5 kg | 0 | 0 | 0 | 35 kg |
| 2023 Dec. 28 | 19:00:02 | 12 kg | 0 | 0 | 2 kg | 0 | 0 | 0 | 35 kg |
| 2023 Dec. 28 | 19:00:03 | 9 kg | 0 | 0 | 1 kg | 0 | 0 | 0 | 35 kg |
| 2023 Dec. 28 | 19:00:04 | 15 kg | 0 | 0 | 1 kg | 0 | 0 | 0 | 0 |
| 2023 Dec. 28 | 20:07:00 | 0 | 0 | 10 kg | 4 kg | 0 | 4 kg | 0 | 33 kg |
| 2023 Dec. 28 | 20:07:01 | 0 | 3 kg | 11 kg | 5 kg | 0 | 5 kg | 0 | 35 kg |
| 2023 Dec. 28 | 20:07:02 | 0 | 0 | 12 kg | 2 kg | 0 | 2 kg | 0 | 35 kg |
| 2023 Dec. 28 | 20:07:03 | 0 | 0 | 9 kg | 1 kg | 0 | 1 kg | 0 | 35 kg |
| 2023 Dec. 28 | 20:07:04 | 0 | 0 | 15 kg | 1 kg | 0 | 1 kg | 0 | 0 |
| 2023 Dec. 30 | 9:55:00 | 10 kg | 0 | 0 | 4 kg | 0 | 0 | 0 | 33 kg |
| 2023 Dec. 30 | 9:55:01 | 11 kg | 3 kg | 0 | 5 kg | 0 | 0 | 0 | 35 kg |
| 2023 Dec. 30 | 9:55:02 | 12 kg | 0 | 0 | 2 kg | 0 | 0 | 0 | 35 kg |
| 2023 Dec. 30 | 9:55:03 | 9 kg | 0 | 0 | 1 kg | 0 | 0 | 0 | 35 kg |
| 2023 Dec. 30 | 9:55:04 | 15 kg | 0 | 0 | 1 kg | 0 | 0 | 0 | 0 |

In Table 2, the grip force is the grip force data A1 generated by the controller 103 after detecting the grip force sensing signal A through the grip force sensing module 1015. The finger forces 1 to 6 correspond to the pressing positions of each of the plurality of finger force buttons (1021a, 1021b, 1021c, 1021d, 1021e, 1021f), and the finger force data C1 generated by the controller 103 after detecting the finger force sensing signal C through the finger force sensing module 1023. The torque is the torque data B1 generated by the controller 103 after detecting the torque sensing signal B through the torque sensing module 1022.

Alternatively, a plurality of case data (e.g., corresponding to different users) could be placed on a single data page, as shown in Table 3.

TABLE 3

| | | | | | finger | finger | finger | finger | finger | |
|---|---|---|---|---|---|---|---|---|---|---|
| Case | | | grip | finger | force | force | force | force | force | |
| No. | Date | Time | force | force1 | 2 | 3 | 4 | 5 | 6 | torque |
| 9527 | 2023 Dec. 28 | 19:00:00 | 10 kg | 0 | 0 | 4 kg | 0 | 0 | 0 | 33 kg |
| 9527 | 2023 Dec. 28 | 19:00:01 | 11 kg | 3 kg | 0 | 5 kg | 0 | 0 | 0 | 35 kg |
| 9527 | 2023 Dec. 28 | 19:00:02 | 12 kg | 0 | 0 | 2 kg | 0 | 0 | 0 | 35 kg |
| 9527 | 2023 Dec. 28 | 19:00:03 | 9 kg | 0 | 0 | 1 kg | 0 | 0 | 0 | 35 kg |
| 9527 | 2023 Dec. 28 | 19:00:04 | 15 kg | 0 | 0 | 1 kg | 0 | 0 | 0 | 0 |
| 007 | 2023 Dec. 28 | 20:07:00 | 0 | 0 | 10 kg | 4 kg | 0 | 4 kg | 0 | 33 kg |
| 007 | 2023 Dec. 28 | 20:07:01 | 0 | 3 kg | 11 kg | 5 kg | 0 | 5 kg | 0 | 35 kg |
| 007 | 2023 Dec. 28 | 20:07:02 | 0 | 0 | 12 kg | 2 kg | 0 | 2 kg | 0 | 35 kg |
| 007 | 2023 Dec. 28 | 20:07:03 | 0 | 0 | 9 kg | 1 kg | 0 | 1 kg | 0 | 35 kg |
| 007 | 2023 Dec. 28 | 20:07:04 | 0 | 0 | 15 kg | 1 kg | 0 | 1 kg | 0 | 0 |
| 9527 | 2023 Dec. 30 | 9:55:00 | 10 kg | 0 | 0 | 4 kg | 0 | 0 | 0 | 33 kg |
| 9527 | 2023 Dec. 30 | 9:55:01 | 11 kg | 3 kg | 0 | 5 kg | 0 | 0 | 0 | 35 kg |
| 9527 | 2023 Dec. 30 | 9:55:02 | 12 kg | 0 | 0 | 2 kg | 0 | 0 | 0 | 35 kg |
| 9527 | 2023 Dec. 30 | 9:55:03 | 9 kg | 0 | 0 | 1 kg | 0 | 0 | 0 | 35 kg |
| 9527 | 2023 Dec. 30 | 9:55:04 | 15 kg | 0 | 0 | 1 kg | 0 | 0 | 0 | 0 |

In Table 3, the grip force is the grip force data A1 generated by the controller 103 after detecting the grip force sensing signal A through the grip force sensing module 1015. The finger forces 1 to 6 correspond to the pressing positions of each of the plurality of finger force buttons (1021a, 1021b, 1021c, 1021d, 1021e, 1021f), and the finger force data C1 generated by the controller 103 after detecting the finger force sensing signal C through the finger force sensing module 1023. The torque is the torque data B1 generated by the controller 103 after detecting the torque sensing signal B through the torque sensing module 1022.

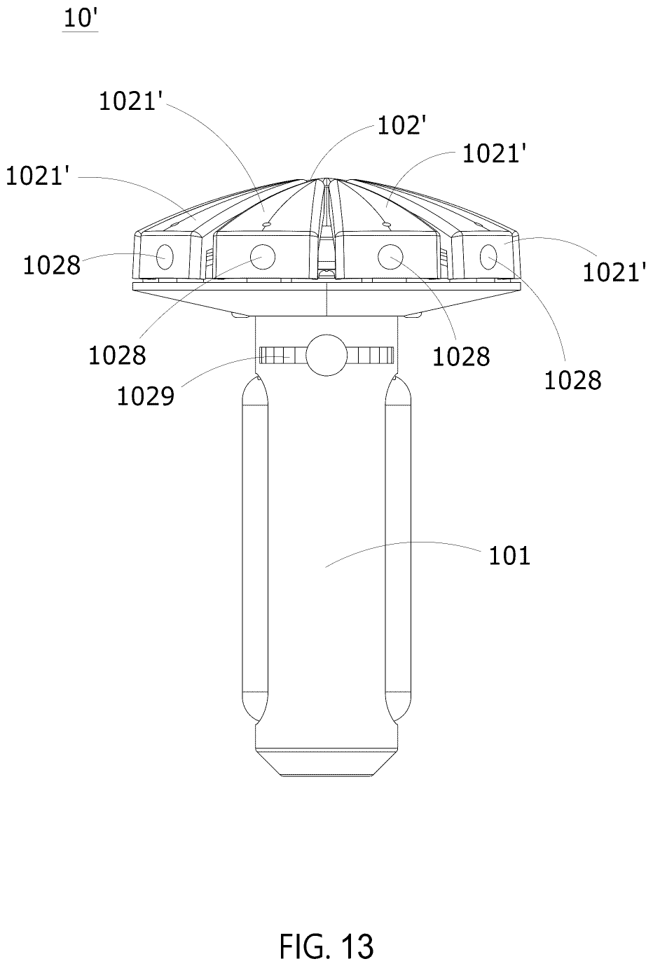

FIG. 13 is a schematic view of the hand rehabilitation evaluation device according to an example implementation of the present disclosure.

Referring to FIG. 13. Each of the plurality of finger force buttons 1021′ of the hand rehabilitation evaluation device 10′ is internally provided with a light-emitting element 1028. When each of the plurality of finger force buttons 1021′ is pressed, the light-emitting element 1028 emits a light source to indicate that the corresponding finger force button 1021′ is being pressed. In some implementations, a brightness or a color of the light-emitting element 1028 changes with the force measured by the plurality of finger force buttons 1021′. For example, the stronger the finger press, the brighter the light, or the stronger the finger press, the color of the light-emitting element 1028 changes. For instance, as the force of the finger press gradually increases, the color of the light-emitting element 1028 will gradually change in sequence from blue, green, yellow, orange, red, and other colors.

Furthermore, a torque indicator light bar 1029 is provided on the exterior of the handle 101′, and the torque indicator light bar 1029 is connected to the controller 103. The torque indicator light bar 1029 could light up according to the rotation direction of the torque dial 1022, providing users with intuitive indication of the torsion amplitude.

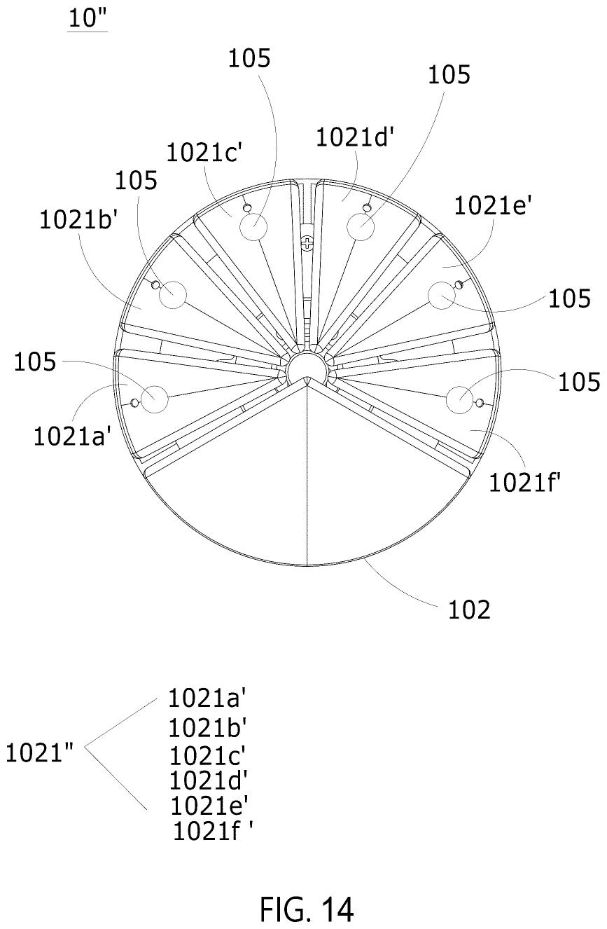

FIG. 14 is a top schematic view of the torque dial according to an example implementation of the present disclosure.

Please also refer to FIG. 5, FIG. 13, and FIG. 14. Unlike the aforementioned hand rehabilitation evaluation devices 10 and 10′ where each of the plurality of finger force buttons (1021a, 1021b, 1021c, 1021d, 1021e, 1021f) on the hand rehabilitation evaluation devices (10, 10′) could be pressed individually, in the hand rehabilitation evaluation device 1″ of an example implementation of the present disclosure, each of the plurality of finger force button 1021″ itself is fixed and immovable, and a finger force push buttons 105 is provided on each of the plurality of finger force button 1021″. Users could place their fingers on the corresponding finger force buttons (1021a′, 1021b′, 1021c′, 1021d′, 1021e′, 1021f′) and press the finger force push button 105 on the finger force button to perform finger force measurement. Each finger force push button 105 is electrically connected to the finger force sensing module 1023. When the finger force push button 105 is subjected to pressure, the finger force sensing module 1023 senses the pressure and generates the corresponding finger force sensing signal C. This design concentrates the force on the finger force push button 105, improving measurement accuracy while simplifying the manufacturing complexity of the overall structure of the torque dial 102″. Additionally, light-emitting elements could also be installed inside each of the finger force push button 105, emitting light when the finger force push button is pressed to provide visual feedback.

In summary, the hand rehabilitation evaluation device of the present disclosure mainly provides functions such as hand torque evaluation, hand grip force evaluation, and finger pressing force evaluation, facilitating hand function evaluation for users or rehabilitation physicians. Through the controller of the present disclosure, sensing signals generated by various sensing modules are received and calculated to produce grip force data, torque data, and finger force data, enabling the present disclosure to achieve convenient evaluation of hand rehabilitation progress and collection of relevant data, serving as a basis and guideline for users or rehabilitation physicians in subsequent rehabilitation treatment. The evaluation of the present disclosure could be applied to the following aspects.

1. As a key indicator of upper limb function and overall health, hand function could be used to evaluate arm function, muscle strength status, and overall health levels. These measurement data reflect not only the strength of the hand but are also related to the overall body condition.

2. Used for disease diagnosis and tracking of functional disabilities, and as a basis for tracking functional improvement during post-surgery or rehabilitation periods.

3. Related to aging, nutrition, and frailty, it could be used for early detection of physical deterioration and even prediction of hospitalization risk and mortality.

4. Used for work and ergonomic design.

The embodiments shown and described above and below are only examples. Many details are often found in the art. Therefore, many such details are neither shown nor described herein for the sake of brevity. Even though numerous characteristics and advantages of the present disclosure have been set forth in the foregoing description, together with details of the structure and function of the present disclosure, the present disclosure is illustrative only, and changes may be made in the details. It will therefore be appreciated that the embodiments described above and below may be modified within the scope of the claims.

What is claimed is:

1. A hand rehabilitation evaluation device, comprising:
a handle comprising a grip force sensing module and a torque sensing module, one side of the handle comprising a first grip actuator and another side of the handle comprising a second grip actuator, each of the first grip actuator and the second grip actuator being connected to the grip force sensing module, the grip force sensing module being configured to generate a grip force sensing signal in response to a grip pressure applied to the first grip actuator and second grip actuator;
a torque dial comprising a first surface rotatably connected to a first end portion of the handle, the torque dial being connected to the torque sensing module, the torque sensing module being configured to generate a torque sensing signal in response to a rotation of the torque dial, the torque dial further comprising a second surface disposed opposite the first surface, the first surface located at a bottom of the torque dial, the second surface being provided with a plurality of finger force buttons, each of the plurality of finger force buttons being coupled to a finger force sensing module, the finger force sensing module being configured to generate a plurality of finger force sensing signals in response to a plurality of pressures applied to the plurality of finger force buttons; and
a controller comprising a processor, the controller being disposed within the handle, the processor electrically connected to the torque sensing module, the grip force sensing module, and the finger force sensing module, the processor being configured to: receive the grip force sensing signal, the torque sensing signal, and the plurality of finger force sensing signals; and generate a grip force data, a torque data, and a finger force data based on the grip force sensing signal, the torque sensing signal, and the plurality of finger force sensing signals.

2. The hand rehabilitation evaluation device of claim 1, wherein the controller further comprises a storage module coupled to the processor, the storage module being configured to store the grip force data, the torque data, and the finger force data generated by the processor.

3. The hand rehabilitation evaluation device of claim 1, wherein the torque sensing module comprises a spring shaft member and a rotary-type variable resistor, the rotary-type variable resistor being disposed within the spring shaft member, when the torque dial rotates relative to the handle, the torque dial is configured to cause the spring shaft member to synchronously rotate with the torque dial, thereby causing a resistance value of the rotary-type variable resistor to change correspondingly with a change of a rotation angle, and the torque sensing module is configured to convert a change of the resistance value into the torque sensing signal, the processor is further configured to calculate a torsion amplitude and a torque based on the torque sensing signal, thereby generating the torque data.

4. The hand rehabilitation evaluation device of claim 1, wherein the finger force sensing module comprises a micro strain gauge-based sensor, each of the plurality of finger force buttons is configured to drive the micro strain gauge-based sensor in response to a pressure applied to a corresponding finger force button of the plurality of finger force buttons, the micro strain gauge-base sensor is configured to sense a pressure conducted by each of the plurality of finger force buttons and to generate the plurality of finger force sensing signals, the finger force sensing module is configured to transmit the plurality of finger force sensing signals to the controller for calculation by the processor to generate the finger force data.

5. The hand rehabilitation evaluation device of claim 4, wherein each of the plurality of finger force buttons is internally provided with a light-emitting element, each of the plurality of finger force buttons is configured to emit a light source in response to the pressure applied to the corresponding finger force button, and a brightness or a color of each of a plurality of light sources emitted, including the light source, by the light-emitting element is determined based on the pressure received by each of the plurality of finger force buttons.

6. The hand rehabilitation evaluation device of claim 1, wherein the grip force sensing module comprises a strain gauge-based sensor, the strain gauge-based sensor is configured to sense the grip pressure in response to the grip pressure applied to the first grip actuator and the second grip actuator to generate the grip force sensing signal, and the grip force sensing module is further configured to transmit the grip force sensing signal to the controller for calculation by the processor to generate the grip force data.

7. The hand rehabilitation evaluation device of claim 2, wherein a second end portion of the handle is provided with a charging transmission port connected to the controller, and the charging transmission port is configured to charge the hand rehabilitation evaluation device and to transmit data.

8. The hand rehabilitation evaluation device of claim 7, wherein the hand rehabilitation evaluation device is detachably connected to a connector, the connector comprises a connection plug, and when the hand rehabilitation evaluation device is connected to the connector, the connection plug engages with the charging transmission port for charging the hand rehabilitation evaluation device and transmitting the data.

9. The hand rehabilitation evaluation device of claim 1, wherein an outer portion of the handle is provided with a display screen and a torque indicator light bar, both of the display screen and the torque indicator light bar are electrically connected to the controller, the display screen is configured to display one of the grip force data, the torque data, and the finger force data, and the torque indicator light bar is configured to illuminate based on a rotation direction of the torque dial.

10. The hand rehabilitation evaluation device of claim 2, wherein the hand rehabilitation evaluation device further comprises:
a wireless transmission module connected to the controller, and the wireless transmission module is configured to externally transmit the grip force data, the torque data, and the finger force data stored in the storage module.

11. The hand rehabilitation evaluation device of claim 8, wherein the connector is provided with a wireless communication module connected to the controller, and the wireless connection module is configured to externally transmit the grip force data, the torque data, and the finger force data stored in the storage module.

12. The hand rehabilitation evaluation device of claim 1, wherein a number of the plurality of finger force buttons is six.
